(19) United States
(12) Patent Application Publication
(10) Pub. No.: US 2026/0229107 A1
(43) Pub. Date: Aug. 6, 2026

(54) FOOD CONSUMPTION REMINDERS

(71) Applicant: Medtronic MiniMed, Inc., Northridge, CA (US)

(72) Inventor: Louis J. LINTEREUR, Boise, ID (US)

(21) Appl. No.: 19/463,456

(22) Filed: Jan. 29, 2026

Related U.S. Application Data
(60) Provisional application No. 63/752,311, filed on Jan. 31, 2025.

(51) Int. Cl. G08B 21/24; G16H 20/60

(52) U.S. Cl. CPC G08B 21/24; G16H 20/60

(57) ABSTRACT

Techniques for providing food consumption reminders are disclosed herein. In some embodiments, the techniques may involve receiving an indication that a patient is to consume a food item after a bolus of insulin is administered. The techniques may further involve determining a duration of time after delivery of the bolus at which the patient is to begin consumption of the food item. The techniques may further involve in response to determining the duration of time has elapsed, causing presentation of a reminder to eat the food item to the patient.

### CROSS-REFERENCE TO RELATED APPLICATION

This application claims the benefit of and priority to U.S. Provisional Application No. 63/752,311, filed Jan. 31, 2025, entitled “FOOD CONSUMPTION REMINDERS,” which is assigned to the assignee hereof and is hereby incorporated by reference in its entirety for all purposes.

### TECHNICAL FIELD

The present disclosure relates generally to food consumption reminders.

### BACKGROUND

People with diabetes are generally instructed to take a bolus of insulin prior to eating, e.g., to mitigate blood glucose rises due to the consumption of food. For example, a patient may ideally bolus insulin ten, 15, etc. minutes prior to eating, which may lead to optimal outcomes in blood glucose level management. However, patients may take a bolus of insulin, and subsequently, forget to eat. This may lead to hypoglycemia, which can be very dangerous.

### SUMMARY

Techniques disclosed herein relate to food consumption reminders. The techniques may be practiced with a processor-implemented method, a system comprising one or more processors and one or more processor-readable media, and/or one or more non-transitory processor-readable media.

According to some embodiments, the techniques may involve receiving an indication that a patient is to consume a food item after a bolus of insulin is administered. The techniques may further involve determining a duration of time after delivery of the bolus at which the patient is to begin consumption of the food item. The techniques may further involve in response to determining the duration of time has elapsed, causing presentation of a reminder to eat the food item to the patient.

According to some embodiments, the techniques may involve determining a duration of time after delivery of a bolus of insulin at which the patient is to begin consumption of a food item based on a nutritional complexity of the food item. The techniques may further involve in response to determining the duration of time has elapsed, causing presentation of a reminder to eat the food item to the patient.

### BRIEF DESCRIPTION OF THE DRAWINGS

The above and other aspects and features of the disclosure will become more apparent in view of the following detailed description when taken in conjunction with the accompanying drawings wherein like reference numerals identify like elements.

FIG. 1 is a diagram of an example of a therapy delivery system, in accordance with aspects of the present disclosure.

FIGS. 2A-2C illustrate example user interfaces in accordance with aspects of the present disclosure.

FIG. 3 is a flowchart of an example process for causing presentation of a reminder to eat in accordance with aspects of the present disclosure.

FIG. 4 is a flowchart of an example process for utilizing a delay duration to present a reminder to eat in accordance with aspects of the present disclosure.

FIG. 5 is a diagram of an example of an insulin delivery device, in accordance with aspects of the present disclosure.

FIG. 6 is a block diagram of an example of a computer system, which can be utilized in embodiments as described herein.

### DETAILED DESCRIPTION

Therapeutic substances (e.g., insulin) may be delivered to a diabetic patient to manage, for example, type I or type II diabetes. Delivering appropriate amounts of a therapeutic substance at appropriate times can help maintain glucose levels within a target range (e.g., a euglycemic range) in the body to prevent hyperglycemia or hypoglycemia conditions.

Patients may take a bolus of insulin prior to eating in order to control for the effects of food consumption on their glucose levels. In particular, a bolus of insulin delivered prior to food consumption may blunt a glucose spike. However, the duration of time between delivery of an insulin bolus and subsequent food consumption that leads to optimal glucose level management varies depending on the type of food being consumed. For example, a food that is primarily composed of carbohydrates, particularly simple carbohydrates such as sugar (e.g., candy, juice, etc.) will typically cause a quick spike in glucose. Accordingly, an insulin bolus should ideally be delivered well before food consumption (e.g., ten minutes, fifteen minutes, etc. prior to consumption of such a food) in order to allow the insulin bolus time to become active in the body prior to consumption of such food. Conversely, a food that is composed of primarily protein or fat, or a mix of protein, fat, and carbohydrates (i.e., a more nutritionally complex food) may cause a more delayed rise in glucose levels. In such cases, the pre-meal bolus of insulin may optimally be delivered soon before food consumption (e.g., concurrent with the start of food consumption, less than five minutes before the start of food consumption, etc.), because the more complex food item(s) cause a slower or more gradual rise in glucose levels.

For various reasons, it may be difficult for a patient to pre-bolus a meal in a manner that leads to optimal glucose level management. For example, in instances in which the insulin bolus should ideally be delivered with a relatively longer duration of time between delivery of the bolus and the start of food consumption (e.g., in instances in which the food to be consumed primarily consists of carbohydrates, particularly simple carbohydrates), the patient may deliver the bolus of insulin, and, while waiting for the duration of time to elapse, forget to consume the food. This may lead to hypoglycemia because the insulin has been delivered without consumption of the food item, which is dangerous for the patient. As another example, it may be difficult for a patient to determine the optimal duration of time to wait to begin food consumption after delivery of an insulin bolus.

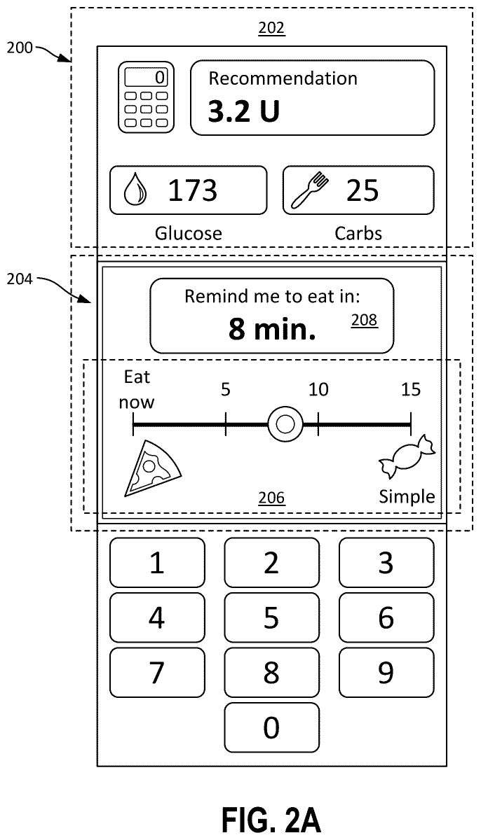

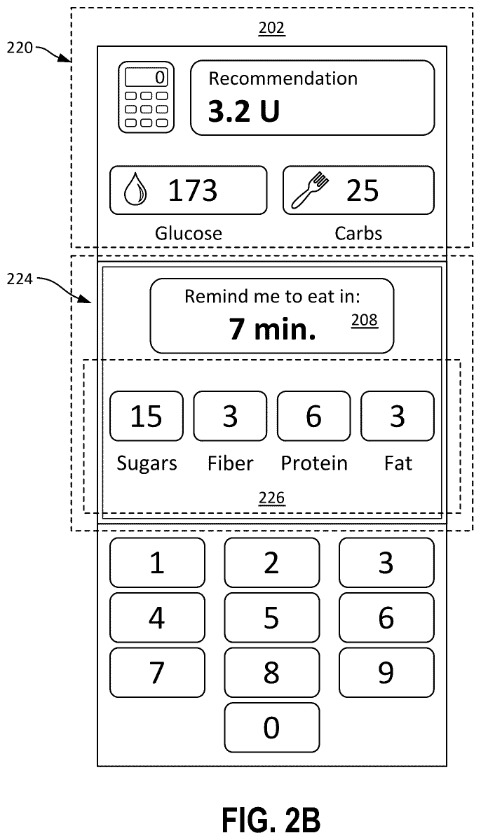

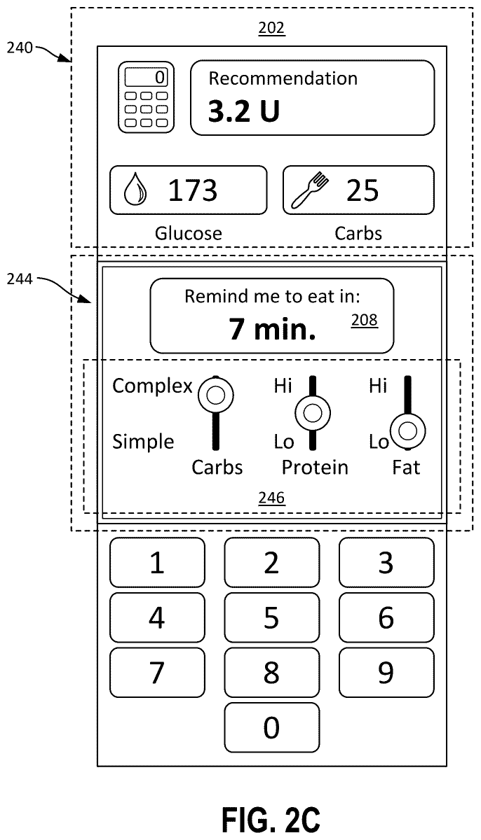

Disclosed herein are techniques for providing more optimal glucose management related to food and/or meal consumption. In particular, the techniques described herein cause a reminder to eat to be presented to the patient after delivery of an insulin bolus. For example, the reminder may include one or more alarms or alerts that remind a patient to consume food item(s) for which a bolus of insulin was previously delivered. Moreover, in some embodiments, the duration of time after which the reminder is to be presented may be determined based on a complexity of the food to be consumed. For example, in an instance in which the patient boluses insulin for planned consumption of a food that is primarily carbohydrates (e.g., juice, candy, etc.), the determined duration of time may be relatively longer (e.g., ten minutes, fifteen minutes, etc.) after delivery of the insulin bolus. Conversely, in an instance in which the patient boluses insulin for planned consumption of a more complex food (e.g., pizza, a soup containing meat and cheese, etc.), the determined duration of time may be relatively shorter (e.g., less than five minutes), or may instruct the patient to immediately begin consuming the food. In some cases, the reminder may be activated and/or set up via a user interface (e.g., as shown in and described below in connection with FIGS. 2A-2C). Such a user interface may be part of a bolus calculator application, e.g., which executes on a personal device and/or an infusion device. Accordingly, via the bolus calculator application, a patient may determine a dosage of insulin to be delivered as a pre-food bolus, and, may additionally confirm and/or configure a reminder to consume the food after a determined duration of time has elapsed.

It should be noted that although the techniques disclosed herein are generally described in connection with an insulin pump as the insulin delivery device, the techniques may be utilized in connection with other delivery devices, such as an insulin pen.

It should be understood that although the techniques described here are generally described in the context of medical devices that provide medical treatment, the techniques may be utilized to transfer therapy state information from a first medical device to a second, replacement medical device for devices which provide treatment for any suitable condition. Examples include pain control devices, hearing devices, etc.

The present disclosure is described primarily with respect to insulin delivery systems. Aspects and embodiments of the present disclosure can be practiced with one or more types of insulin (e.g., fast-acting insulin, intermediate-acting insulin, and/or slow-acting insulin). For example, fast-acting insulin may be used for both basal dosages and bolus dosages.

Although the present disclosure is described primarily with respect to insulin delivery systems, the scope of the present disclosure is not limited to insulin delivery systems. Rather, the present disclosure applies to and can be implemented for other therapy systems as well. For example, some techniques of the present disclosure may be adapted for practice in relation to glucagon delivery systems.

Discussions utilizing terms such as, for example, “processing,” “computing,” “calculating,” “determining,” “establishing,” “analyzing,” “checking,” or the like, may refer to operation(s) and/or process(es) of a computer, a computing platform, a computing system, or other electronic computing device, that manipulate and/or transform data represented as physical (e.g., electronic) quantities within the computer's registers and/or memories into other data similarly represented as physical quantities within the computer's registers and/or memories or other non-transitory information storage media that may store instructions to perform operations and/or processes by, e.g., one or more processor or processor apparatus (e.g., system on a chip) or a device associated with such processor(s).

In the context of this disclosure, a “module” may refer to a set of computer-executable instructions and/or a hardware processor configured to execute a set of computer-executable instructions. A hardware processor may be an integrated circuit device associated with a computing device, such as a server or a user device (e.g., a desktop computer, a laptop computer, a tablet computer, a mobile phone, or the like), which is programmable to perform specific tasks. In some embodiments, multiple modules may be implemented as a single module. In some embodiments, a single module may be implemented as multiple modules. In some embodiments, two or more modules may be executable by the same device (e.g., the same computing device or delivery device).

Unless explicitly stated, the methods described herein are not constrained to a particular order or sequence. Additionally, some of the described methods or elements thereof can occur or be performed simultaneously or concurrently.

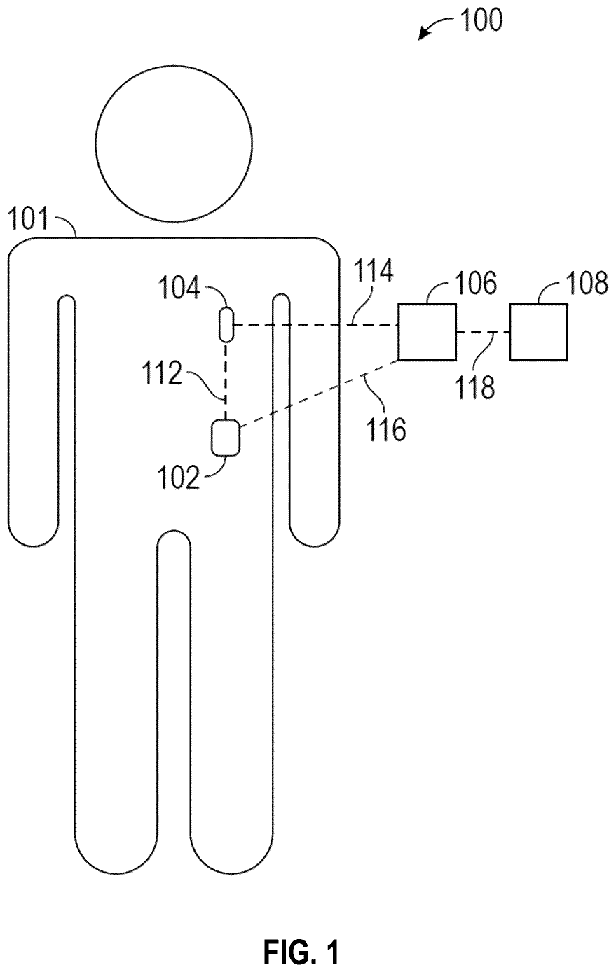

FIG. 1 depicts an example therapy delivery system 100 for a person 101. Components of therapy delivery system 100 may be used to implement one or more blocks of process 300 and/or process 400. System 100 may be an insulin delivery system. The depicted therapy delivery system 100 includes a delivery device 102, a monitoring device 104, a computing device 106, and an optional remote or cloud computing system 108. The delivery device 102, the monitoring device 104, and the computing device 106 may be embodied in various ways, including being disposed in one or more device housings. For example, in some embodiments, all of the devices 102-106 may be disposed in a single device housing. In some embodiments, each of the devices 102-106 may be disposed in a separate device housing. In some embodiments, two or more of the devices 102-106 may be disposed in the same device housing, and/or a single device 102, 104, or 106 may have two or more parts that are disposed in two or more housings. Such embodiments, and combinations thereof, are contemplated to be within the scope of the present disclosure. Note that the locations of each device shown in FIG. 1 is merely one example. For example, monitoring device 104 is depicted on a chest of the patient, however, in some implementations, monitoring device 104 may be on an arm of the patient.

FIG. 1 also depicts communications links 112-118. The communications links 112-118 may each be a wired connection and/or a wireless connection. In the case where two devices are located in the same device housing, the communication link may include, for example, wires, cables, and/or communication buses on a printed circuit board, among other things. In the case where two devices are separated from each other in different device housings, the communication links may be wired and/or wireless connections. Wired connections may include, without limitation, an Ethernet connection, a USB connection, and/or another type of physical connection. Wireless connections may include, without limitation, a cellular connection, a Wi-Fi connection, a Bluetooth® connection, a mesh network connection, and/or another type of connection using a wireless communication protocol. Some embodiments of the communication links 112-118 may use direct connections, such as Bluetooth® connections, and/or may use connections that route through one or more networks or network devices (not shown), such as an Ethernet network, a Wi-Fi network, a cellular network, a satellite network, an intranet, an extranet, the Internet, and/or the Internet backbone, among other types of networks. Various combinations of wired and/or wireless connections may be used for the communication links 112-118.

Aspects of the insulin delivery system 100 are described below. Further aspects and details may be described in U.S. Pat. Nos.: 4,562,751; 4,685,903; 5,080,653; 5,505,709; 5,097,122; 6,485,465; 6,554,798; 6,558,320; 6,558,351; 6,641,533; 6,659,980; 6,752,787; 6,817,990; 6,932,584; and 7,621,893. The entire contents of each of the foregoing United States Patents are hereby incorporated by reference herein.

The delivery device 102 is configured to deliver a therapeutic substance (e.g., insulin) to a person 101. The delivery device 102 may be secured to the person 101 (e.g., to the body or clothing of the person 101) or may be implanted on or in the body of the person 101. In some embodiments, the delivery device 102 may include a reservoir, an actuator, a delivery mechanism, and a cannula (not shown). The reservoir may be configured to store an amount of the therapeutic substance. In some embodiments, the reservoir may be refillable or replaceable. The actuator may be configured to drive the delivery mechanism. In some examples, the actuator may include a motor, such as an electric motor. The delivery mechanism may be configured to move the therapeutic substance from the reservoir through the cannula. In some examples, the delivery mechanism may include a pump and/or a plunger. The cannula may facilitate a fluidic connection between the reservoir and the body of the person 101. The cannula and/or a needle may facilitate delivery of the therapeutic substance to a tissue layer, vein, or body cavity of the person 101. During operation, the actuator, in response to a signal (e.g., a command signal), may drive the delivery mechanism, thereby causing the therapeutic substance to move from the reservoir, through the cannula, and into the body of the person 101.

The components of the delivery device 102 described above are merely provided as examples. The delivery device 102 may include other components, such as, without limitation, a power supply, a communication transceiver, computing resources, and/or user interfaces, among other things. Persons skilled in the art will recognize various implementations of the delivery device 102 and the components of such implementations. All such implementations and components are contemplated to be within the scope of the present disclosure.

With continuing reference to FIG. 1, the monitoring device 104 is configured to detect a physiological condition (e.g., a glucose concentration level) of the person 101 and may also be configured to detect other things. The monitoring device 104 may be secured to the body of the person 101 (e.g., to the skin of person 101 via an adhesive) and/or may be at least partially implanted into the body of the person 101. Depending on the particular location or configuration, the monitoring device 104 may be in contact with biological matter (e.g., interstitial fluid and/or blood) of the person 101.

The monitoring device 104 includes one or more sensors (not shown), such as, without limitation, electrochemical sensors, electrical sensors, and/or optical sensors. As persons skilled in the art will understand, an electrochemical sensor may be configured to respond to the interaction or binding of a biological marker to a substrate by generating an electrical signal based on a potential, conductance, and/or impedance of the substrate. The substrate may include a material selected to interact with a particular biomarker, such as glucose. The potential, conductance, and/or impedance may be proportional to a concentration of the particular biomarker. In the case of electrical sensors, and as persons skilled in the art will understand, an electrical sensor may be configured to respond to an electrical biosignal by generating an electrical signal based on an amplitude, frequency, and/or phase of the electrical biosignal. The electrical biosignal may include a change in electric current produced by the sum of an electrical potential difference across a tissue, such as the nervous system, of the person 101. In some embodiments, the electrical biosignal may include portions of a potential change produced by the heart of the person 101 over time, e.g., recorded as an electrocardiogram, that are indicative of a glucose level of the person 101. In the case of optical sensors, as persons skilled in the art will understand, an optical sensor may be configured to respond to the interaction or binding of a biological marker to a substrate by generating an electrical signal based on change in luminance of the substrate. For example, the substrate may include a material selected to fluoresce in response to contact with a selected biomarker, such as glucose. The fluorescence may be proportional to a concentration of the selected biomarker.

In some embodiments, the monitoring device 104 may include other types of sensors that may be worn, carried, or coupled to the person 101 to measure activity of the person 101 that may influence the glucose levels or glycemic response of the person 101. As an example, the sensors may include an acceleration sensor configured to detect an acceleration of the person 101 or a portion of the person 101, such as the person's hands or feet. The acceleration (or lack thereof) may be indicative of exercise, sleep, or food/beverage consumption activity of the person 101, which may influence the glycemic response of the person 101. In some embodiments, the sensors may include heart rate and/or body temperature, which may indicate an amount of physical exertion experienced by the person 101. In some embodiments, the sensors may include a GPS receiver which detects GPS signals to determine a location of the person 101.

The sensors described above are merely provided as examples. Other sensors or types of sensors for monitoring physiological condition, activity, and/or location, among other things, will be recognized by persons skilled in the art and are contemplated to be within the scope of the present disclosure. For any sensor, the signal provided by a sensor shall be referred to as a “sensor signal.”

The monitoring device 104 may include components and/or circuitry configured to pre-process sensor signals. Pre-processing may include, without limitation, amplification, filtering, attenuation, scaling, isolation, normalization, transformation, sampling, and/or analog-to-digital conversion, among other things. Persons skilled in the art will recognize various implementations for such pre-processing, including, without limitation, implementations using processors, controllers, ASICS, integrated circuits, hardware, firmware, programmable logic devices, and/or machine-executable instructions, among others. The types of pre-processing and their implementations are merely provided as examples. Other types of pre-processing and implementations are contemplated to be within the scope of the present disclosure. In some embodiments, the monitoring device 104 may not perform pre-processing.

As used herein, the term “sensed data” shall mean and include the information represented by a sensor signal or by a pre-processed sensor signal. In some embodiments, sensed data may include glucose levels in a person 101, acceleration of a part of the person 101, heart rate of the person 101, temperature of the person 101, and/or geolocation (e.g., GPS location) of the person 101, among other things. The monitoring device 104 may communicate sensed data to the delivery device 102 via communication link 112 and/or to the computing device 106 via communication link 114. Use of sensed data by the delivery device 102 and/or by the computing device 106 will be described later herein.

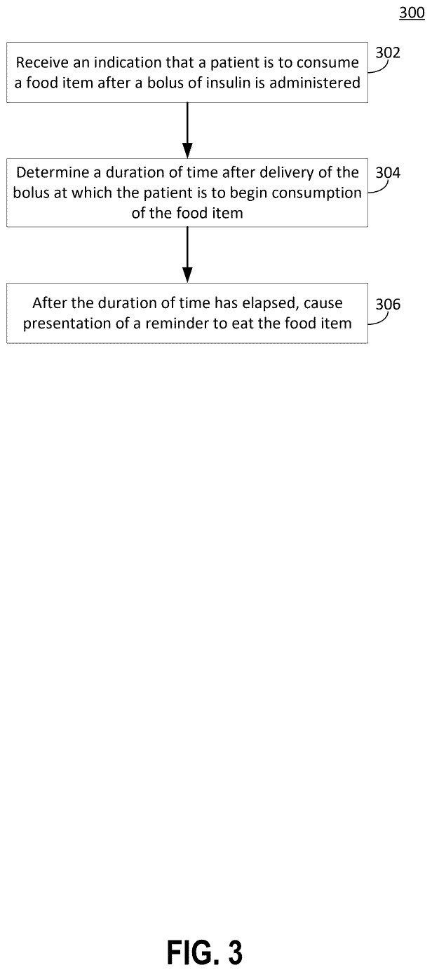

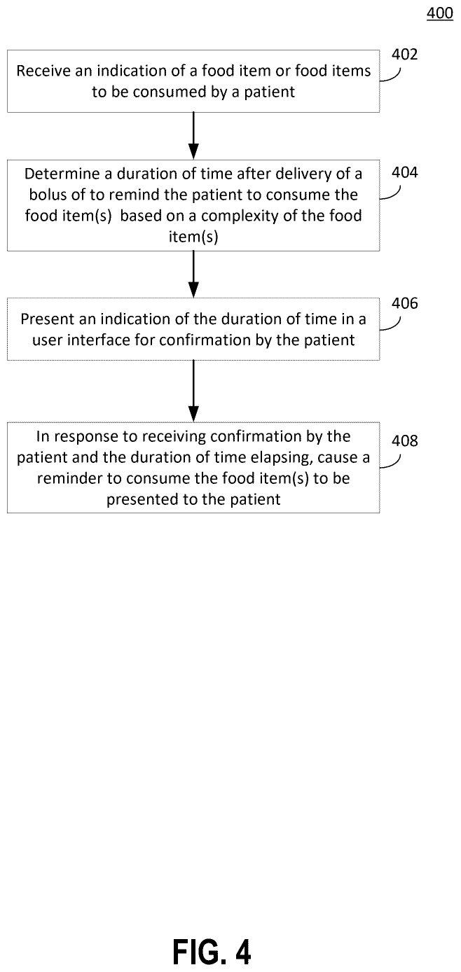

The computing device 106 provides processing capabilities and may be implemented in various ways. In some embodiments, the computing device 106 may be a consumer device, such as a smartphone, a computerized wearable device (e.g., a smartwatch), a tablet computer, a laptop computer, or a desktop computer, among others, or may be a special purpose device (e.g., a portable control device) provided by, for example, the manufacturer of the delivery device 102. In some embodiments, the computing device 106 may be “processing circuitry” (defined below) that is integrated with another device, such as the delivery device 102. In some embodiments, the computing device 106 may be secured to the person 101 (e.g., to the body or clothing of person 101), may be at least partially implanted into the body of person 101, and/or may be held by the person 101. In some embodiments, computing device 106 may be configured to execute one or more blocks of process 300 and/or process 400, shown in FIGS. 3 and 4 below. For example, in some embodiments, computing device 106 may determine a CIR for a patient based on insulin dosage metrics.

For each of the embodiments of the computing device 106, the computing device 106 may include various types of logic circuitry, including, but not limited to, microprocessors, microcontrollers, digital signal processors (DSPs), application specific integrated circuits (ASICs), field-programmable gate arrays (FPGAs), central processing units (CPU), graphics processing units (GPU), programmable logic devices, memory (e.g., random access memory, volatile memory, non-volatile memory, etc.), or other discrete or integrated logic circuitry, as well as combinations of such components. The term “processing circuitry” may generally refer to any of the foregoing logic circuitry, alone or in combination with other logic circuitry, or any other circuitry for performing computations.

Aspects of the delivery device 102, the monitoring device 104, and the computing device 106 have been described above. One or more of the devices 102-106 may include a user interface (not shown) that presents information to the person 101 and/or receives information from the person 101. The user interface may include a graphical user interface (GUI), a display device, a keyboard, a touchscreen, a speaker, a microphone, a vibration motor, buttons, switches, and/or other types of user interfaces. Persons skilled in the art will recognize various types of user interfaces that may be used, and all such user interfaces are contemplated to be within the scope of the present disclosure. For example, where the computing device 106 is a consumer device such as a smart phone, tablet computer, laptop computer, or the like, the user interfaces would include a display device, a physical and/or virtual keyboard, and/or audio speakers provided by such consumer devices, among other things. In some embodiments, a user interface may notify the person 101 of sensed data (e.g., glucose level) and/or insulin delivery data (e.g., rates of historic, current, or future insulin delivery) and may present alerts to the person 101. In some embodiments, a user interface may receive inputs from the person 101, which may include, for example, a requested change in insulin delivery and/or a meal indication, among other things. The descriptions and embodiments above regarding user interfaces are merely provided as examples, and other types and other uses of user interfaces are contemplated to be within the scope of the present disclosure.

The following describes communications between the devices 102-106 and cooperation between the devices 102-106 with respect to insulin delivery. As depicted in FIG. 1, and as mentioned above, the devices 102-106 may communicate with each other via communication links 112-116. In some embodiments, the computing device 106 may control operation of the delivery device 102 and/or the monitoring device 104. For example, the computing device 106 may generate one or more signals (e.g., a command signal) that cause the delivery device 102 to deliver insulin to the person 101, e.g., as a basal dosage and/or a bolus dosage. In some embodiments, the computing device 106 may receive data associated with insulin delivery (e.g., insulin delivery data) from the delivery device 102 and/or receive sensed data (e.g., glucose levels) from the monitoring device 104 and may perform computations based on the insulin delivery data, the sensed data, and/or other data to control the delivery device 102. Insulin delivery data may include, but is not limited to, a type of insulin being delivered, historical insulin delivery rates and/or amounts, current insulin delivery rate and/or amount, and/or user input affecting insulin delivery. As persons skilled in the art will understand, in a closed-loop operating mode, computing device 106 may communicate dosage commands to the delivery device 102 based on a difference between a current glucose level in the body of the person 101 (e.g., received from the monitoring device 104) and a target glucose level (e.g., determined by the computing device 106). The dosage commands may indicate an amount of insulin to be delivered and/or a rate of insulin delivery and may regulate the current glucose level toward the target glucose level. Examples of closed-loop operations for insulin infusion systems are described in U.S. Pat. Nos. 6,088,608, 6,119,028, 6,589,229, 6,740,072, 6,827,702, 7,323,142, and 7,402,153, and in United States Patent Application Publication Nos.: 2014/0066887 and 2014/0066889. The entire contents of each of the foregoing patents and publications are hereby incorporated by reference herein.

With continuing reference to FIG. 1, the remote or cloud computing system 108 may be a proprietary remote/cloud computing system or a commercial cloud computing system including one or more server computing devices. The remote/cloud computing system 108 may provide additional computing resources on-demand as needed when the computing resources of a client computing device (e.g., the computing device 106) are not sufficient. The computing device 106 and the remote/cloud computing system 108 may communicate with each other through a communication link 118, which may traverse one or more communication networks (not shown). The communication networks may include, without limitation, an Ethernet network, Wi-Fi network, a cellular network, a satellite network, an intranet, an extranet, the Internet, and/or the Internet backbone, among other types of networks. Persons skilled in the art will recognize implementations for the remote/cloud computing system 108 and how to interface with such systems through various types of networks. For example, the remote/cloud computing system 108 may include an array of processing circuitry (defined above) and may execute machine-readable instructions. Such implementations, interfaces, and networks are contemplated to be within the scope of the present disclosure. In some embodiments, remote or cloud computing system 108 may be configured to execute one or more blocks of process 300 and/or process 400, as shown in FIGS. 3 and 4, respectively. For example, in some embodiments, remote or cloud computing system 108 may be configured to determine a CIR for a patient. In some embodiments, remote or cloud computing system 108 may store and/or analyze data from a population of patients used to determine model parameters for a model that relates insulin dosage metrics to a CIR. In some embodiments, remote or cloud computing system 108 may determine the model parameters for the model. In some embodiments, remote or cloud computing system 108 may store a look-up table that includes results from the model, e.g., that indicates CIRs for different values of an insulin dosage metric. In some such embodiments, remote or cloud computing system 108 may transmit any suitable information, such as data from a look-up table, model parameters, and/or a determined CIR to computing system associated with a particular medical device (e.g., computing system 106) for use with the medical device.

An example therapy delivery system has been described above. For convenience, the description below may primarily refer to an insulin delivery system as an example of the therapy delivery system. However, it is intended that any aspect, embodiment, or description relating to an insulin delivery system shall be applicable to a therapy delivery system which delivers a therapy other than insulin.

In some embodiments, a user interface may be presented which allows a user to configure a duration of time after which a reminder to eat will be presented. The user interface may be part of a bolus calculator interface. Such a bolus calculator interface may calculate a recommended bolus dosage. For example, the bolus dosage may be calculated based on nutritional information of a food to be consumed (e.g., based on the amount of carbohydrates, fats, and/or proteins to be consumed). In some implementations, the user interface may suggest a recommended duration of time after delivery of the bolus at which the user should consume the food for optimal control of glucose levels. The duration of time may be calculated based on nutritional information of the food to be consumed. For example, the duration of time may be calculated based on the complexity of the food. Complexity may represent the balance of carbohydrates to protein and/or fat, whether the carbohydrates are considered “simple” or “complex,” the amount of fiber, etc. In some cases, complexity information may be input by the user via the user interface. The user interface may present a suggested duration of time after which the reminder is to be presented (e.g., representing an optimal duration of time to eat the food after taking the bolus dosage). In some implementations, the user may be prompted, via the user interface, to confirm the suggested duration of time. Confirmation of the suggested duration of time may cause an alert timer to be initiated for the duration of time. In some embodiments, the user interface may allow the user to modify the suggested duration of time, e.g., to make the duration of time longer or shorter. In some embodiments, the user interface may allow the user to configure a manner in which the reminder is delivered. For example, the reminder may be delivered as a push notification on a mobile device or wearable device, with haptic feedback, with an audible and/or visual alert, etc.

FIG. 2A illustrates an example user interface 200 in accordance with some embodiments. As illustrated, user interface 200 includes a bolus calculation region 202. Bolus calculation region 202 may include user interface controls (e.g., text boxes, drop down boxes, etc.) in which a user can input nutritional information, etc., from which a bolus dosage recommendation is calculated as presented. Note that bolus calculation region 202 may be associated with a bolus calculator application, e.g., which executes on a mobile device or other patient device paired with an insulin pump, or which executed on an insulin pump.

As illustrated, user interface 200 additionally includes a reminder configuration region 204. Reminder configuration region 204 includes a nutritional complexity input 206. In the example shown in FIG. 2A, nutritional complexity input 206 includes a slider control configured to receive user input regarding the complexity of the food the patient is planning to consume. In the example shown in FIG. 2A, the left side of the slider corresponds to relatively complex foods (e.g., that include a mix of carbohydrates, proteins, and fats), such as pizza, and the right side corresponds to relatively simple foods (e.g., that include mostly simple carbohydrates such as glucose), such as candy. As indicated by the two sides of the slider input, it may be optimal to consume more complex foods immediately or soon after taking the insulin bolus, and conversely, it may be optimal to consume more simple foods after waiting a relatively longer duration of time (e.g., 10 minutes, 15 minutes, 20 minutes, etc.) after taking the insulin bolus.

Reminder configuration region 204 includes a reminder confirmation button 208. Reminder confirmation button 208 indicates the suggested duration of time to wait after taking the insulin bolus to eat. Selection of confirmation button 208 may activate triggering of a reminder to eat after the duration of time indicated (e.g., 8 minutes in the example shown in FIG. 2A) has passed after delivery of the insulin bolus.

FIG. 2B illustrates another example user interface 220 in accordance with some embodiments. User interface 220 is similar to what is shown in and described above in connection with FIG. 2A, however, user interface 220 utilizes a different nutritional complexity input 226 within reminder configuration region 224. In the example shown in FIG. 2B, nutritional complexity input 226 receives indications of amounts of various macronutrients in the food to be consumed (e.g., in grams). The macronutrients may include sugar and fiber (e.g., to indicate the complexity of the carbohydrates to be consumed), protein, and/or fat. Utilizing the macronutrient information provided in nutritional complexity input 226, a duration of time to wait after delivery of the bolus may be determined. The duration of time is presented within confirmation button 208, similar to what is described above in connection with FIG. 2A.

FIG. 2C illustrates another example user interface 240 in accordance with some embodiments. User interface 240 is similar to what is shown in and described above in connection with FIGS. 2A and 2B, however, user interface 240 utilizes a different nutritional complexity input 246 within reminder configuration region 244. In the example shown in FIG. 2C, nutritional complexity input 246 receives indications of the amount and/or types of various macronutrients using sliders (rather than the numerical input shown in FIG. 2B). For example, nutritional complexity input 246 includes a first slider associated with carbohydrates that receives input regarding whether the carbohydrates in the food to be consumed are more simple, more complex, or a balance of simple and complex. Nutritional complexity input 246 includes second and third slider inputs for receiving amounts of protein and fat, respectively. By not requiring numerical input as in FIG. 2B, a user may be able to more quickly indicate the nutritional complexity of the food to be consumed without providing exact values, which may be faster for the user. Utilizing the macronutrient information provided in nutritional complexity input 246, a duration of time to wait after delivery of the bolus may be determined. The duration of time is presented within confirmation button 208, similar to what is shown in and described above in connection with FIG. 2A.

As described above in connection with FIGS. 2A-2C, in some embodiments, a duration of time after delivery of a bolus of insulin at which a patient is to begin consumption of a food item may be determined. The duration of time after bolus delivery may be determined based on nutritional information. For example, the duration of time may be determined such that the duration of time between delivery of the bolus and when the patient is to consume the food item is shorter for food items determined to be more nutritionally complex (e.g., containing a mix of protein, fat, and carbohydrates), for food items determined to have more complex carbohydrates compared to simple carbohydrates (e.g., having more than a predetermined threshold amount of fiber, having more fiber than sugar, etc.), based on the amount of carbohydrates, or the like. The nutritional information may be provided by user input (e.g., as shown in the user interfaces of FIGS. 2A-2C), or may be determined by accessing a food library. For example, determining nutritional information using a food library may involve querying a food library (e.g., stored in a database) with an indication of one or more type(s) of food to be consumed and receiving, from the food library, indications of the nutritional information. The type(s) of food may include categories of food (e.g., bread, fruit, cereal, etc.), specific types of food and/or preparations of food (e.g., grilled chicken, white bread, apple, popcorn, buttered popcorn, etc.), or the like. In some cases, the food library may be queried with a specific brand of food, e.g., using a scanned barcode and/or based on user input that specifies the specific brand of food. In some cases, the food library may receive, as input, portion information, where the nutritional information may be determined based on a combination of the type(s) of food to be consumed and the portion information. In some embodiments, the food library may be populated with default nutritional values for various types of food, various brands of food, etc. The default values may be scaled based on portion information to determine the nutritional information used to determine duration of time after delivery of the bolus at which the patient is to begin consuming the food. The food library may store default values in, e.g., a look-up table to determine values of fats, proteins, carbohydrates, etc. Alternatively, in some embodiments, the food library may store an effect of consumption of the food on glucose levels, and the nutritional information may be estimated based on the effect on glucose levels (e.g., using a regression function or the like).

In some implementations, a duration of time at which to present a reminder to eat the food item may be confirmed by the patient. In other words, a confirmation may be requested from the patient to set an alarm that will be triggered upon expiration of the duration of time, as shown in and described above in connection with FIGS. 2A-2C. Upon expiration of the duration of time, an alarm may be presented which reminds the patient to eat the food item. The alarm may include haptic feedback, visual feedback (e.g., a visual alert), audible feedback, or any combination thereof. The alarm may be presented via an insulin pump, and/or via a paired device, such as a mobile phone, a wearable device (e.g., a smart watch), or the like.

FIG. 3 is a flowchart of an example process 300 for causing presentation of a food consumption reminder in accordance with some embodiments. Blocks of process 300 may be executed by one or more processors, e.g., of an infusion device, a mobile device or personal device, a wearable device, or the like. In some implementations, blocks of process 300 may be executed in an order other than what is shown in FIG. 3. In some embodiments, two or more blocks of process 300 may be executed substantially in parallel. In some embodiments, one or more blocks of process 300 may be omitted.

Process 300 can begin at 302 by receiving an indication that a patient is to consume a food item after a bolus of insulin is determined and/or delivered. The indication may be received via a bolus calculator application, e.g., that executes on a mobile device or an infusion device. The indication may include a type of food the patient is planning to consume. For example, the type of food may include a name of the food (e.g., “pizza,” “ice cream,” “potato chips,” etc.). In some embodiments, the indication may include nutritional information associated with the food. For example, the nutritional information may include a portion size to be consumed, macronutrient information (e.g., the amount of protein, fat, carbohydrates, fiber, etc.), a relative complexity of carbohydrates in the food (e.g., whether the food contains more simple carbohydrates relative to complex carbohydrates, amounts of sugar and/or fiber, or the like), etc. Nutritional information may be obtained via user input. For example, the user input may be received via user interface controls where a user may indicate the complexity of the food, macronutrient information, etc. The user interface controls may include one or more sliders (e.g., as shown in and described above in connection with FIG. 2C), one or more radio buttons (e.g., to select from a closed set of options, such as whether the protein content is high or low, whether the carbohydrates or simple or complex, etc.), one or more drop-down menus (e.g., to select from a closed set of options, such as whether the protein content is high or low, whether the carbohydrates or simple or complex, etc.), one or more dials, text boxes to receive open-ended input from the user, a camera interface which may allow a user to scan a barcode of a package food item to be consumed, etc. Note that a user interface may include multiple types of user interface controls.

Additionally or alternatively, in some embodiments, nutritional information may be obtained by accessing a food library to obtain nutritional information associated with a particular food item or food items. As described above, determining nutritional information using a food library may involve querying a food library (e.g., stored in a database) with an indication of one or more type(s) of food to be consumed and receiving, from the food library, indications of the nutritional information. The type(s) of food may include categories of food (e.g., bread, fruit, cereal, etc.), specific types of food and/or preparations of food (e.g., grilled chicken, white bread, apple, popcorn, buttered popcorn, etc.), or the like. In some cases, the food library may be queried with a specific brand of food, e.g., using a scanned barcode and/or based on user input that specifies the specific brand of food. In some cases, the food library may receive, as input, portion information, where the nutritional information may be determined based on a combination of the type(s) of food to be consumed and the portion information. In some embodiments, the food library may be populated with default nutritional values for various types of food, various brands of food, etc. The default values may be scaled based on portion information to determine the nutritional information used to determine duration of time after delivery of the bolus at which the patient is to begin consuming the food. The food library may store default values in, e.g., a look-up table to determine values of fats, proteins, carbohydrates, etc. Alternatively, in some embodiments, the food library may store an effect of consumption of the food on glucose levels, and the nutritional information may be estimated based on the effect on glucose levels (e.g., using a regression function or the like).

At 304, process 300 can determine a duration of time after delivery of the bolus at which the patient is to begin consumption of the food item. In some implementations, the duration of time may be determined based on the nutritional complexity of the food item. More detailed techniques for determining the duration of time are shown in and described below in connection with FIG. 4.

At 306, process 300 can, after the duration of time has elapsed, cause presentation of a reminder to eat the food item. The reminder may be presented as an alarm. The reminder may be associated with haptic feedback (e.g., vibration), audible feedback (e.g., a spoken message, beeps or other noises, etc.), and/or visual feedback (e.g., a message reminding the patient to eat). The reminder may be presented via the infusion device, a mobile device paired with the infusion device, and/or a wearable device paired with the infusion device and/or the mobile device. Note that, in some implementations, a manner of presentation of the reminder (e.g., which device presents the reminder, types of feedback used to present the reminder, etc.) may be configured by the patient, e.g., in a “Settings” portion of a bolus calculator application.

As described above, in some embodiments, a duration of time after delivery of an insulin bolus after which to remind the patient to consume a particular food item or food items may be determined. The determined duration of time may be one that is optimal for glucose management. For example, the determined duration of time may be one that maximizes a duration of time that the patient has their glucose levels withing a target range (e.g., between 70 mg/dl-120 mg/dl, or the like). In some implementations, the duration of time may be determined based on a complexity of the food item(s) the patient is planning to consume, based on nutritional information, based on portion size, etc. In other words, the duration of time may be one that optimizes glucose level control based on a time a bolus of a given dosage is delivered in relation to the food that is to be consumed. The duration of time may be recommended to the patient. For example, the patient may be asked to confirm that a reminder to eat should be presented after the duration of time has elapsed. In some cases, the patient may confirm that the reminder is to be presented, may modify the duration of time, etc.

Note that, in some embodiments, process 300 may monitor the patient to confirm the patient began consumption of the food item(s). Monitoring the patient may involve monitoring glucose level trends over time to verify that changes in glucose levels conform to expected or projected levels of glucose given provision of the insulin bolus and consumption of the food item(s) after the determined duration of time has elapsed between delivery of the bolus and consumption of the food item(s). For example, in some embodiments, monitoring glucose level trends over time may involve comparing a measured glucose level to a threshold and/or comparing a determined rate of change of glucose levels to a threshold. Continuing with this example, process 300 may determine that the patient has begun food consumption responsive to determining the glucose level exceeds a threshold glucose level and/or that a rate of change of the glucose level exceeds a rate of change threshold. Conversely, in some embodiments, process 300 may determine that the patient has not begun consuming the food item(s) responsive to determining the glucose level is below a threshold glucose level and/or that a rate of change of the glucose level is below a rate of change threshold. In some cases, responsive to determining that glucose level trends do not conform to expected values, process 300 may deliver a further prompt or reminder.

Note that, in some embodiments, it may be determined that the patient has begun consuming the food prior to delivery of the reminder to begin eating. Determining that the patient has begun consuming food may be based on monitoring glucose levels (e.g., by comparing glucose level and/or rate of change of glucose level to predetermined thresholds as described above). Responsive to determining the patient has begun consuming the food item(s) prior to delivery of the reminder alert, the reminder alert may be automatically (e.g., without user input) cleared. As a specific example, in an instance in which the reminder alert is set for 10 minutes, and it is determined that the patient begins eating 8 minutes after delivery of the bolus, the reminder may be cleared after determining the patient has begun eating and prior to the reminder alert being delivered after 10 minutes. Automatically clearing the reminder may avoid annoying the patient with unnecessary alerts.

FIG. 4 is a flowchart of an example process 400 for determining a duration of time after which a reminder to eat is to be presented and delivering such a reminder in accordance with some embodiments. Blocks of process 400 may be executed by one or more processors, e.g., of an infusion device, a mobile device, a wearable device, or the like. In some implementations, blocks of process 400 may be executed in an order other than what is shown in FIG. 4. In some embodiments, two or more blocks of process 400 may be executed substantially in parallel. In some embodiments, one or more blocks of process 400 may be omitted.

Process 400 can begin at 402 by receiving an indication of a food item or food items to be consumed by a patient. The indication may be received via an application, e.g., a bolus calculator application, which executes on an infusion device or a personal device (e.g., a mobile phone). The indication may include a meal composed of multiple different food items (e.g., a soup that includes various vegetables, meats, etc.), a set of individual food items that compose a meal (e.g., a salad, a bread roll, and a meat), an individual food item (e.g., a piece of candy, popcorn, etc.), or any combination thereof. The indication may include portion size(s). In some implementations, the indication may include nutrition information, e.g., macronutrient information.

At 404, process 400 can determine a duration of time after delivery of a bolus to remind the patient to consume the food item(s) based on a complexity of the food item(s). As described above, the duration of time may be based on nutritional content of the food item(s). For example, food item(s) containing a balance of carbohydrates, proteins, and/or fats or that contain little carbohydrates may have a relatively shorter duration of time between delivery of the insulin bolus and when the patient should eat relative to food item(s) that contain primarily carbohydrates. In general, the duration of time may be determined to align the timing of the onset of insulin action with the expected rise in blood glucose due to consumption of the food item(s). The onset of insulin action for rapid acting insulins may be around, e.g., 15-20 minutes. The time when glucose begins to rise following eating depends on meal type/nutritional content. For example, a meal consisting of simple carbohydrates (e.g., juice, candy, white bread, etc.) may cause a nearly immediate rise in blood glucose, and accordingly, the bolus should be delivered well ahead of eating (e.g., 15-20 minutes prior to eating). Conversely, more complex meals will take longer to cause a rise in blood glucose, and accordingly, the bolus may be delivered closer to the time of eating.

As a specific example, process 400 may determine a duration of time within a range of about 10-20 minutes responsive to determining the patient is planning to consume one or more pieces of candy. As another specific example, process 400 may determine a duration of time within a range of about 8-15 minutes responsive to determining the patient is planning to consume potato chips. As another example, food item(s) containing relatively more complex carbohydrates (e.g., whole grains, food with relatively high amounts of fiber, etc.) may have a relatively shorter duration of time between delivery of the insulin bolus and when the patient should eat relative to food item(s) that contain relatively more simple carbohydrates (e.g., that are primarily sugar, refined flour, etc.). As a specific example, process 400 may determine a duration of time within a range of about 0-5 minutes responsive to determining the patient is planning to consume a slice of pizza. As another specific example, process 400 may determine a duration of time within a range of about 0-5 minutes responsive to determining the patient is planning to consume a salad with a source of protein.

In some implementations, the duration of time may be determined by providing nutrition information to a trained model (e.g., a trained machine learning model) or utilizing nutrition information as inputs to a mathematical function or equation that generates the duration of time as an output. In some implementations, a look-up table may be used that relates one or more nutritional information metrics to a duration of time. The nutrition information may include the number of carbohydrates to be consumed, a metric that quantifies the complexity of the food item(s) based on a balance of carbohydrates, proteins, and fats and/or the complexity of the carbohydrates, or the like. Note that, in instances in which a trained machine learning model is used, the model may be trained based on training data that includes nutrition information and an optimal duration of time between delivery of the insulin and consumption of the food item such that the model is trained to predict the duration of time. Such a model may be trained using data from the patient and/or data from people other than the patient. In some cases, a mathematical function and/or a machine learning model may be updated over time based on data from the patient. For example, in an instance in which a duration of time is generated, and the patient consumed the food item after the duration of time has elapsed, the trend of the glucose levels of the patient after consumption of the food item after the duration of time has elapsed may be analyzed. In instances in which the glucose levels, e.g., leave a target range, a model or mathematical function may be adapted to provide a different duration of time in future instances of consumption of that food item.

Note that, in instances in which a machine learning model is used, the machine learning model may have any suitable type of architecture (e.g., a neural network, a deep neural network, a support vector machine, a logistic regression, a classifier, etc.). The model may be trained in accordance with the type of architecture used. For example, model architectures which generate, as output, a classification from a closed set of classifications may be trained to generate an output corresponding to a duration of time between bolus and initiation of food consumption from a closed set of time durations. For example, such a model may be trained to take, as input, nutritional information, and generate, as an output, a selection of a duration of time from, e.g., the candidate durations of time of 0 minutes (e.g., “eat now”), 5 minutes, 10 minutes, and 15 minutes. In other examples, a machine learning model may generate, as an output, a continuous valued duration of time given input nutritional information.

At 406, process 400 can present an indication of the duration of time in a user interface for confirmation by the patient. Examples of a user interface for confirmation of the duration of time are shown in and described above in connection with FIGS. 2A, 2B, and 2C. For example, as shown in FIGS. 2A, 2B, and 2C, user interface control 208 requests that a user confirm that a reminder for the determined duration of time is to be presented.

At 408, process 400 can, in response to receiving confirmation by the patient and the duration of time elapsing (e.g., after delivery of the bolus of insulin), cause a reminder to consume the food item(s) to be presented to the patient. As described above in connection with block 306 of FIG. 3, the reminder may include any suitable haptic feedback, visual feedback, audio feedback, etc., and may be presented by any suitable device or combination of devices.

### Example Apparatus

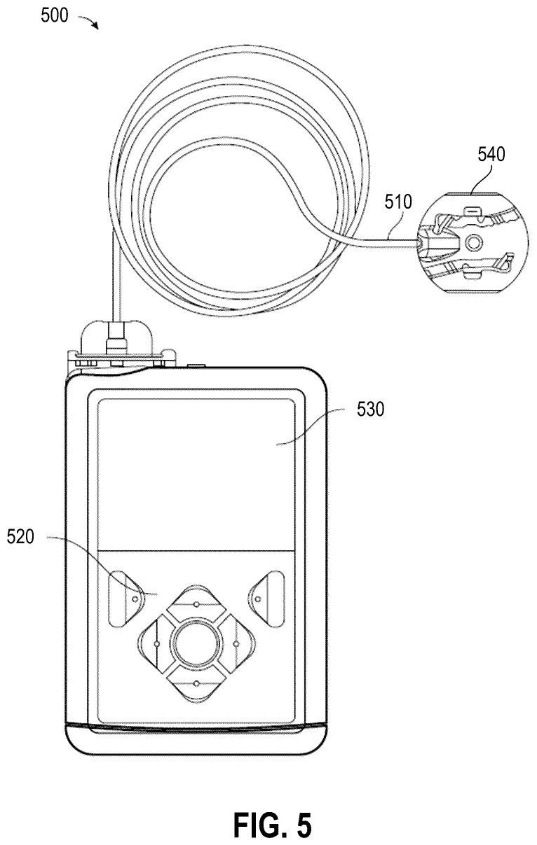

In some embodiments, therapy may be effected based on communicating a therapy determination toward a therapy delivery device. A non-limiting example of such a device is described below in connection with FIG. 5, which depicts an example insulin delivery device 500, in accordance with aspects of the present disclosure.

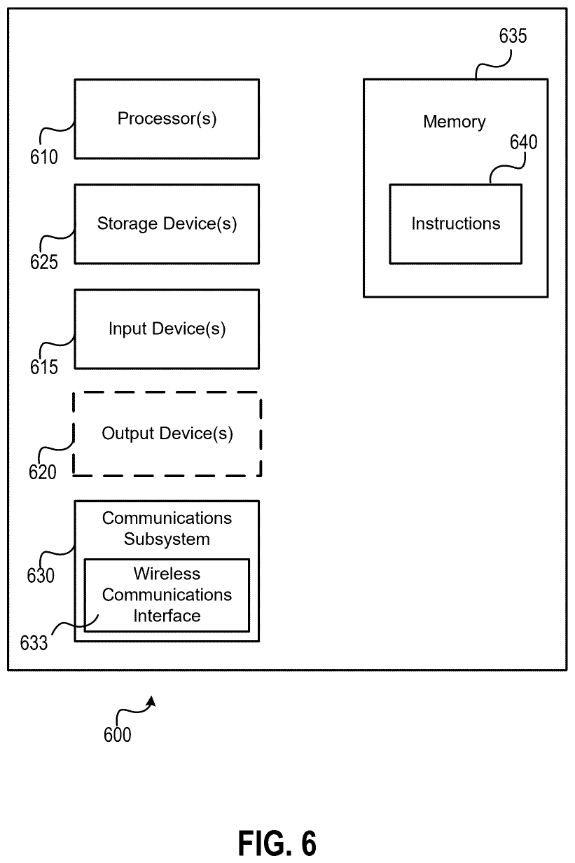

As mentioned above, therapy determinations may be communicated toward an insulin delivery device 500 (e.g., from a cloud computing system 108 via an intermediary computing device 106 communicatively coupled to the device 500). The insulin delivery device 500 may be an example of the delivery device 102 as described throughout this disclosure. In such a device, insulin delivery may be performed based on internal communication between a central computing module (e.g., a microcontroller for device 500 as a whole) and an insulin delivery module (e.g., including a motor and a pump). For instance, insulin delivery may be caused by the central computing module communicating a delivery command in the form of an electrical signal that travels via a communication fabric to the insulin delivery module. The central computing module may also be configured to communicate (e.g., via a transceiver) with a computing device (e.g., 106, FIG. 1) communicatively coupled to a remote or cloud computing system (e.g., 108, FIG. 1). The insulin delivery device 500 may communicate various event data (e.g., meal data, exercise data, and/or insulin delivery data) toward the remote or cloud computing system, which may communicate insulin delivery determinations toward the insulin delivery device 500, in some implementations. FIG. 6 further illustrates components which can be included in the insulin delivery device 500.

The insulin delivery device 500 can provide fast-acting insulin through a small tube 510 configured for fluidic connection with a cannula (not shown). The cannula may be inserted subcutaneously under a fixation dressing 540 that includes an inlet for the tube 510, an outlet for the cannula, and an adhesive surface for affixing the dressing 540 to skin. The device 500 can deliver at least two types of dosages—a basal dosage, which can be delivered periodically (e.g., every five minutes) in tiny amounts throughout the day and night, and a bolus dosage to cover an increase in blood glucose from meals and/or to otherwise correct high blood glucose levels. The depicted insulin delivery device 500 includes a user interface having button elements 520 that can be manipulated to administer a bolus of insulin, to change therapy settings, to change user preferences, to select display features, and the like. The insulin delivery device 500 also includes a display device 530 that can be used to present various types of information or data to the user (such as a notification or an alert of the type described above). In accordance with aspects of the present disclosure, a user of the insulin delivery device 500 may use the button elements 520 to input certain event data (e.g., event type, event start time, event details, etc.), and the user inputs can be confirmed using the display device 530. The depicted insulin delivery device 500 of FIG. 5 is merely provided by way of example, and other types of insulin delivery devices and other techniques different from those described above are contemplated to be within the scope of the present disclosure.

FIG. 6 is a block diagram of an embodiment of a computer system 600, which can be utilized in embodiments as described herein. It should be noted that FIG. 6 is meant only to provide a generalized illustration of various components, any or all of which may be utilized as appropriate. In addition, it can be noted that components illustrated by FIG. 6 can be localized to a single device (e.g., delivery device 102, monitoring device 104, computing device 106, or computing system 108) and/or distributed among various networked devices, which may be disposed at different geographical locations.

In some embodiments, the computer system 600 may include hardware elements that can be electrically coupled via a bus (or may otherwise be in communication, as appropriate). The hardware elements may include processor(s) 610, which may comprise, without limitation, one or more microcontroller(s), one or more microprocessor(s), one or more general-purpose processors, one or more special-purpose processors (such as digital signal processing chips, graphics acceleration processors, and/or the like), and/or other processing structure, which can be configured to perform one or more of the methods or functionalities described herein.

In some embodiments, the computer system 600 also may include one or more input devices 615, which may comprise, without limitation, button elements 520, a microphone, a glucose sensor, and/or the like. The computer system 600 may also include one or more output devices 620, which may comprise without limitation a display device (e.g., 530), a speaker, a buzzer, and/or the like.

In some embodiments, the computer system 600 may further include one or more non-transitory storage devices 625, which can include, without limitation, local and/or network accessible storage, and/or may comprise, without limitation, a disk drive, a drive array, an optical storage device, a solid-state storage device, such as a read-access memory (RAM) and/or read-only memory (ROM), which can be programmable, flash-updateable, and/or the like. Such storage devices may be configured to implement any appropriate data stores, including without limitation, various file systems, database structures, and/or the like. Such data stores may include database(s) and/or other data structures used to store and administer messages and/or other information to be sent to one or more other components or external devices.

In some embodiments, the computer system 600 may also include a communications subsystem 630, which may implement wireless communication technologies managed and controlled by a wireless communication interface 633. Additionally or alternatively, communications subsystem 630 may implement wired technologies (such as Ethernet, coaxial communications, universal serial bus (USB), or the like). The wireless communication interface 633 may comprise one or more wireless transceivers that may send and receive wireless signals (e.g., signals according to Bluetooth, Bluetooth Low Energy (BLE)). Thus, the communications subsystem 630 may comprise a modem, a network card (wireless or wired), an infrared communication device, a wireless communication device, and/or a chipset, and/or the like, which may enable the computer system 600 to communicate with any device discussed with respect to FIG. 1, including delivery device 102, monitoring device 104, computing device 106, and/or a cloud computing system 108 as described herein. Hence, the communications subsystem 630 may be used to receive and send data (e.g., SG, Ip, insulin delivery information) as described in the embodiments herein.

In some embodiments, the computer system 600 will further comprise a working memory 635, which may comprise a RAM or ROM device, as described above. Software elements, shown as being located within the working memory 635, may comprise computer-readable and computer-executable instructions 640; device drivers; executable libraries; and/or other code, which may comprise computer programs used in various embodiments and/or may be designed to implement methods and/or configure systems in accordance with embodiments described herein. Merely by way of example, one or more operations described with respect to the methods or functionalities discussed above might be implemented as code and/or instructions executable by a computer (and/or a processor within a computer). Such code and/or instructions can be used to configure and/or adapt a general-purpose computer (or other device) to perform one or more operations in accordance with the described methods.

In some embodiments, a set of these instructions and/or code may be stored on a non-transitory computer-readable storage medium, such as the storage device(s) 625 described above. In some cases, the storage medium might be incorporated within a computer system, such as computer system 600. In other embodiments, the storage medium might be separate from a computer system (e.g., a removable medium, such as an optical disc) and/or provided in a downloadable installation package, such that the storage medium can be used to program, configure, and/or adapt a general purpose computer with the instructions and/or code stored thereon. These instructions might take the form of executable code, which is executable by the computer system 600, and/or might take the form of source and/or installable code, which, upon compilation and/or installation on the computer system 600 (e.g., using any of a variety of generally available compilers, installation programs, compression/decompression utilities, etc.), then takes the form of executable code.

The embodiments disclosed herein are examples of the disclosure and may be embodied in various forms. For instance, although certain embodiments herein are described as separate embodiments, each of the embodiments herein may be combined with one or more of the other embodiments herein. Specific structural and functional details disclosed herein are not to be interpreted as limiting, but as a basis for the claims and as a representative basis for teaching one skilled in the art to variously employ the present disclosure in virtually any appropriately detailed structure. Like reference numerals may refer to like elements throughout the description of the figures.

Any of the herein described techniques, operations, methods, programs, algorithms, or codes may be converted to, or expressed in, a programming language or computer program embodied on a computer, processor, or machine-readable medium. The terms “programming language” and “computer program,” as used herein, each include any language used to specify instructions to a computer or processor, and include (but is not limited to) the following languages and their derivatives: Assembler, Basic, Batch files, BCPL, C, C+, C++, Delphi, Fortran, Java, JavaScript, machine code, operating system command languages, Pascal, Perl, PL1, Python, scripting languages, Visual Basic, metalanguages which themselves specify programs, and all first, second, third, fourth, fifth, or further generation computer languages. Also included are database and other data schemas, and any other meta-languages. No distinction is made between languages which are interpreted, compiled, or use both compiled and interpreted approaches. No distinction is made between compiled and source versions of a program. Thus, reference to a program, where the programming language could exist in more than one state (such as source, compiled, object, or linked) is a reference to any and all such states. Reference to a program may encompass the actual instructions and/or the intent of those instructions.

It should be understood that the foregoing description is only illustrative of the present disclosure. To the extent consistent, any or all of the aspects detailed herein may be used in conjunction with any or all of the other aspects detailed herein. Various alternatives and modifications can be devised by those skilled in the art without departing from the disclosure. Accordingly, the present disclosure is intended to embrace all such alternatives, modifications, and variances. The embodiments described with reference to the attached drawing figures are presented only to demonstrate certain examples of the disclosure. Other elements, steps, methods, and techniques that are insubstantially different from those described above and/or in the appended claims are also intended to be within the scope of the disclosure.

While several embodiments of the disclosure have been depicted in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

### Example Embodiments

Embodiment 1: A method, comprising: receiving an indication that a patient is to consume a food item after a bolus of insulin is administered; determining a duration of time after delivery of the bolus at which the patient is to begin consumption of the food item; and in response to determining the duration of time has elapsed, causing presentation of a reminder to eat the food item to the patient.

Embodiment 2: The method of embodiment 1, wherein determining the duration of time is based on a nutritional complexity of the food item.

Embodiment 3: The method of embodiment 2, further comprising: receiving an indication of macronutrient content of the food item; and determining the nutritional complexity of the food based on the macronutrient content.

Embodiment 4: The method of embodiment 3, further comprising obtaining the macronutrient content of the food item from a food library.

Embodiment 5: The method of embodiment 3, wherein the macronutrient content is received as user input via a user interface.

Embodiment 6: The method of embodiment 5, wherein the user interface comprises one or more controls to enter values of a corresponding one or more macronutrients of the food item.

Embodiment 7: The method of any one of embodiments 1-6, further comprising presenting a user interface that requests patient confirmation that a reminder is to be presented after the determined duration of time has elapsed, wherein causing presentation of the reminder is additionally in response to the patient confirmation.

Embodiment 8: The method of any one of embodiments 1-7, wherein the determined duration of time is specified and/or modified by the patient.

Embodiment 9: The method of any one of embodiments 1-8, further comprising: receiving a second indication that a patient is to administer a second bolus prior to consumption of a second food item; and determining timing of presentation of a second reminder to eat the second food item based at least in part on timing of the reminder to eat the food item and a complexity of the second food item.

Embodiment 10: A system comprising: one or more processors; and one or more processor-readable media storing instructions. When executed by one or more processors, the instructions cause performance of: receiving an indication that a patient is to consume a food item after a bolus of insulin is administered; determining a duration of time after delivery of the bolus at which the patient is to begin consumption of the food item; and in response to determining the duration of time has elapsed, causing presentation of a reminder to eat the food item to the patient.

Embodiment 11: The system of embodiment 10, wherein determining the duration of time is based on a nutritional complexity of the food item.

Embodiment 12: The system of embodiment 11, wherein the instructions further cause performance of: receiving an indication of macronutrient content of the food item; and determining the nutritional complexity of the food based on the macronutrient content.

Embodiment 13: The system of embodiment 12, wherein the instructions further cause performance of obtaining the macronutrient content of the food item from a food library.

Embodiment 14: The system of embodiment 12, wherein the macronutrient content is received as user input via a user interface.

Embodiment 15: The system of embodiment 14, wherein the user interface comprises one or more controls to enter values of a corresponding one or more macronutrients of the food item.

Embodiment 16: The system of any one of embodiments 10-15, wherein the instructions further cause performance of presenting a user interface that requests patient confirmation that a reminder is to be presented after the determined duration of time has elapsed, wherein causing presentation of the reminder is additionally in response to the patient confirmation.

Embodiment 17: The system of any one of embodiments 10-16, wherein the determined duration of time is specified and/or modified by the patient.

Embodiment 18: The system of any one of embodiments 10-17, wherein the instructions further cause performance of: receiving a second indication that a patient is to administer a second bolus prior to consumption of a second food item; and determining timing of presentation of a second reminder to eat the second food item based at least in part on timing of the reminder to eat the food item and a complexity of the second food item.

Embodiment 19: A method, comprising: determining a duration of time after delivery of a bolus of insulin at which the patient is to begin consumption of a food item based on a nutritional complexity of the food item; and in response to determining the duration of time has elapsed, causing presentation of a reminder to eat the food item to the patient.

Embodiment 20: The method of embodiment 19, further comprising receiving an indication of the nutritional complexity via a user interface associated with a bolus calculator application.

What is claimed is:

1. A method, comprising:
receiving an indication that a patient is to consume a food item after a bolus of insulin is administered;
determining a duration of time after delivery of the bolus at which the patient is to begin consumption of the food item; and
in response to determining the duration of time has elapsed, causing presentation of a reminder to eat the food item to the patient.

2. The method of claim 1, wherein determining the duration of time is based on a nutritional complexity of the food item.

3. The method of claim 2, further comprising:
receiving an indication of macronutrient content of the food item; and
determining the nutritional complexity of the food based on the macronutrient content.

4. The method of claim 3, further comprising obtaining the macronutrient content of the food item from a food library.

5. The method of claim 3, wherein the macronutrient content is received as user input via a user interface.

6. The method of claim 5, wherein the user interface comprises one or more controls to enter values of a corresponding one or more macronutrients of the food item.

7. The method of claim 1, further comprising presenting a user interface that requests patient confirmation that a reminder is to be presented after the determined duration of time has elapsed, wherein causing presentation of the reminder is additionally in response to the patient confirmation.

8. The method of claim 1, wherein the determined duration of time is specified and/or modified by the patient.

9. The method of claim 1, further comprising:
receiving a second indication that a patient is to administer a second bolus prior to consumption of a second food item; and
determining timing of presentation of a second reminder to eat the second food item based at least in part on timing of the reminder to eat the food item and a complexity of the second food item.

10. A system comprising:
one or more processors; and
one or more processor-readable media storing computer-readable instructions which,
when executed by one or more processors, cause performance of:
receiving an indication that a patient is to consume a food item after a bolus of insulin is administered;
determining a duration of time after delivery of the bolus at which the patient is to begin consumption of the food item; and
in response to determining the duration of time has elapsed, causing presentation of a reminder to eat the food item to the patient.

11. The system of claim 10, wherein determining the duration of time is based on a nutritional complexity of the food item.

12. The system of claim 11, wherein the instructions further cause performance of:
receiving an indication of macronutrient content of the food item; and
determining the nutritional complexity of the food based on the macronutrient content.

13. The system of claim 12, wherein the instructions further cause performance of obtaining the macronutrient content of the food item from a food library.

14. The system of claim 12, wherein the macronutrient content is received as user input via a user interface.

15. The system of claim 14, wherein the user interface comprises one or more controls to enter values of a corresponding one or more macronutrients of the food item.

16. The system of claim 10, wherein the instructions further cause performance of presenting a user interface that requests patient confirmation that a reminder is to be presented after the determined duration of time has elapsed, wherein causing presentation of the reminder is additionally in response to the patient confirmation.

17. The system of claim 10, wherein the determined duration of time is specified and/or modified by the patient.

18. The system of claim 10, wherein the instructions further cause performance of:
receiving a second indication that a patient is to administer a second bolus prior to consumption of a second food item; and
determining timing of presentation of a second reminder to eat the second food item based at least in part on timing of the reminder to eat the food item and a complexity of the second food item.

19. A method, comprising:
determining a duration of time after delivery of a bolus of insulin at which a patient is to begin consumption of a food item based on a nutritional complexity of the food item; and
in response to determining the duration of time has elapsed, causing presentation of a reminder to eat the food item to the patient.

20. The method of claim 19, further comprising receiving an indication of the nutritional complexity via a user interface associated with a bolus calculator application.
